(19) United States
(12) Patent Application Publication
(10) Pub. No.: US 2025/0099415 A1
(43) Pub. Date: Mar. 27, 2025

(54) Methods for Inhibiting the Progression of Oxidative Retinal Disease

(71) Applicant: BioJiva LLC, Los Altos, CA (US)

(72) Inventors: Karsten Schmidt, Los Altos, CA (US); Mikhail Shchepinov, Oxford (GB)

(73) Assignee: BioJiva LLC, Los Altos, CA (US)

(21) Appl. No.: 18/291,016

(22) PCT Filed: Jul. 22, 2022
(86) PCT No.: PCT/US22/38072

§ 371 Date: Jan. 22, 2024

Related U.S. Application Data
(60) Provisional application No. 63/224,674, filed on Jul. 22, 2021.
(60) Provisional application No. 63/224,679, filed on Jul. 22, 2021.
(60) Provisional application No. 63/224,690, filed on Jul. 22, 2021.

(51) Int. Cl. A61K 31/202; A61K 31/232; A61P 27/02

(52) U.S. Cl. CPC A61K 31/202; A61K 31/232; A61P 27/02

(57) ABSTRACT

Disclosed are methods for inhibiting the progression of oxidative retinal diseases. The methods include administration of deuterated docosahexaenoic acid or an ester thereof to a patient in need thereof.

### CROSS-REFERENCE TO RELATED APPLICATIONS

This application claims priority to U.S. Provisional Patent Application Ser. No. 63/224,690, filed on Jul. 22, 2021; U.S. Provisional Patent Application Ser. No. 63/224,679, filed on Jul. 22, 2021; and U.S. Provisional Patent Application Ser. No. 63/224,674, filed on Jul. 22, 2021, each of which is incorporated herein by reference in its entirety.

### TECHNICAL FIELD

Disclosed are methods for inhibiting the progression of oxidative retinal diseases in humans. The methods include a dosing regimen to treat a patient suffering from a neurodegenerative ocular disease treatable with a deuterated docosahexaenoic acid (DHA) or a prodrug thereof. In particular, the dosing regimen provides for rapid onset to a therapeutic concentration in vivo of deuterated DHA at a level where the progression of the disease is reduced notwithstanding the gradual increase in metabolic uptake of this compound in the treated patient.

### BACKGROUND

There are many oxidative retinal diseases in humans which are largely incurable, lead to visual impairment and, in too many cases, blindness. Typically, when diagnosed early, the attending physician instructs the patient to quit smoking, establish a healthy lifestyle and take vitamins and/or antioxidants in order to slow the rate of disease progression. See, e.g., mayoclinic.org/diseases-conditions/dry-macular-degeneration/diagnosis-treatment/drc-20350381.

Recent advances in the understanding of the underlying etiology of these diseases have implicated oxidative stress as a significant component. However, ocular inflammation, age, and the immune system have also been identified as contributing factors. See, e.g., Knickelbein, et al., Int. Ophthalmol. Clin., 2015:55(3)63-78.

Despite years of research and an understanding of the underlying etiology, many if not most oxidative retinal diseases remain difficult to treat. For example, the current standard of treatment for macular degeneration includes periodic intraocular injections of anti-VEGF antibodies. See, e.g., Moutray, et al., Ther. Adv. Chron. Dis. 2(5):325-311 (2011). However, the fact that such treatment requires intraocular injections limits its widespread use. Accordingly, there exists a need for new treatments of oxidative retinal diseases, including macular degeneration. Preferably, such new treatments would be non-invasive and even more preferably, could be administered orally.

### SUMMARY

The retina contains very high levels of docosahexaenoic acid which is found in the highest concentrations in the disk membranes of the outer segments of photoreceptor cells including rods that help convert light into electrical and chemical signals for the brain. Docosahexaenoic acid accounts for most of the total polyunsaturated fatty acid groups present in the phospholipids of rod's outer segment membranes of the photoreceptor cells, This represents a proportion higher than is found in any other tissues in the human body.

Peroxidation of docosahexaenoic acid occurs in the retina and, in particular, in the rods and is due to an imbalance between routine production and subsequent detoxification of reactive oxygen species (“ROS”). Docosahexaenoic acid (DHA) has the structure:

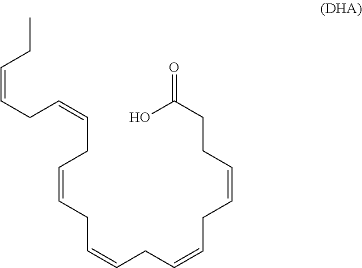 [SMILES: CC/C=C\C/C=C\C/C=C\C/C=C\C/C=C\C/C=C\CCC(=O)O]

and is a 22-carbon chain omega-3 polyunsaturated fatty acid (“PUFA”) having 6 sites of cis-unsaturation. Separating each of these 6 sites are 5 bis-allylic methylene groups. These groups are particularly susceptible to oxidative damage due to ROS. Moreover, due to the stacking nature in the rods, oxidation of a bis-allylic position in a first DHA leads to an oxidative cascade of neighboring DHAs known as lipid auto-peroxidation (LPO). This cascade generates significant damage to the retina and negatively affects the viability of the retina. In addition, the oxidized DHAs lead to oxidation of membrane proteins as well as being converted into a large number of highly reactive carbonyl compounds. The ongoing imbalance in the oxidative process leads to continued degradation in the retina of the patient's eyes.

Recently, Shchepinov, U.S. Pat. No. 10,058,522, disclosed that oxidative retinal diseases are treatable by administration of deuterated docosahexaenoic acid or an ester thereof. After administration, a portion of the deuterated docosahexaenoic acid is incorporated into the retina, including the rods, thereby stabilizing these rods against oxidative damage. Such stabilization is due to the enhanced stability of a carbon-deuterium bond as opposed to a carbon-hydrogen bond. However, the time required to achieve a therapeutic concentration of deuterated docosahexaenoic acid in the retina is extensive and is measured in months. This is due to the fact that replacement of non-deuterated DHA in the rods with deuterated DHA is gradual which leads to a substantial period between start of therapy and the generation of a therapeutic level of deuterated DHA in the retina. Of course, during this period, the patient's ocular disease progresses with concomitant loss of visual functionality.

As above, many retinal diseases involve progressive deterioration of the patient's vision due to the uncheck pathology of the disease. For example, macular degeneration initially manifests itself with dimming and distortion of the patient's vision, followed by further deterioration which ultimately leads to blindness. Given the progressive nature of these diseases coupled with the goal of maintaining as much of the patient's vision as possible for as long as possible, it is desirable to achieve a therapeutic concentration of docosahexaenoic acid in vivo as quickly as possible. However, the dosing of any deuterated DHA is complicated by the several factors. These include, as examples, the body's limitation as to how much polyunsaturated fatty acids, including deuterated DHA, can be absorbed per day; the variable intake of PUFAs by each patient on a day-to-day basis that often exceeds the maximum amount of PUFAs that can be absorbed; the variability of PUFA absorption on a patient-by-patient basis; patient compliance; and patients with conditions that interfere with PUFA uptake (e.g., C. difficile infections and the antibiotic associated diarrhea that accompanies treatment).

All of the above evidence an ongoing need to provide for a dosing regimen that allows for the rapid uptake of deuterated docosahexaenoic acid into the body and particularly the retina that can be universally applied to patients having different metabolism, body mass, and degree of retinal degeneration due to the disease.

In one embodiment, this disclosure provides for dosing protocols that allow for the uptake of deuterated docosahexaenoic acid or an ester thereof in amounts that provide for an accelerated onset of a therapeutic concentration in the retina and reduction in the rate of disease progression. In one embodiment, such a reduction is based on the differential in the extent of the disease progression in treated patients as compared to patients treated with placebo control at 6 months, or at 12 months, or at 18 months, or at 24 months interval between initiation of therapy and evaluation of disease progression. In one embodiment, this differential in the extent of disease progression in treated patients as compared to patients treated with placebo is about 20% as measured by reduced geographic atrophy expansion over a 6 or 12, or 18, or 24 month interval between initiation of therapy and evaluation of disease progression.

In one embodiment, a dosing protocol is provided which comprises the periodic administration of a unit dose of deuterated docosahexaenoic acid or an ester thereof per day. The unit dose is selected to provide for an accelerated (rapid) uptake of the deuterated docosahexaenoic acid in the retina. The unit dose can be split into 1, 2, 3 or 4 subunits each administered on the same day.

In one embodiment, there is provided a method for treating an oxidative retinal disease in a patient in need thereof said method comprises the periodic administration to said patient of from about 100 mg/day to about 1,000 mg/day of a composition comprising a deuterated docosahexaenoic acid or ester thereof wherein said administration results in a therapeutic concentration of deuterated docosahexaenoic acid in the retina coupled with a reduction in the rate of progression of said oxidative retinal disease. In one embodiment, the periodic dosing to the patient is from about 100 mg/day to about 350 mg/day. In another embodiment, the periodic dosing to the patient is from about 350 mg/day to about 650 mg/day. In yet another embodiment, the periodic dosing to the patient is from about 650 mg/day to about 1.000 mg/day. In some cases, the periodic dosing to the patient can range from about 100 mg/day to about 1,250 mg/day.

The methods described herein provide for an accelerated onset of a therapeutic concentration of deuterated docosahexaenoic acid in vivo to minimize unnecessary loss of vision functionality in the treated patients suffering from an oxidative retinal disease.

In one embodiment, said periodic administration of the unit dose comprises administration for at least 5 days per week and preferably 7 days a week.

In one embodiment, said periodic administration of the unit dose comprises administration for at least about 70% of the days per month and preferably at least about 80% of the days per month.

In one embodiment, the deuterated docosahexaenoic acid ester is a C₁-C₆ alkyl ester and preferably an ethyl ester.

In one embodiment, the per day dosing of the deuterated docosahexaenoic acid or ester thereof is about 100 mg/day; or about 125 mg/day; or about 150 mg/day; or about 175 mg/day; or about 200 mg/day; or about 225 mg/day; or about 250 mg/day; or about 275 mg/day; or about 300 mg/day; or about 325 mg/day; or about 350 mg/day; or about 375 mg/day, or about 400 mg/day; or about 425 mg/day; or about 450 mg/day; or about 475 mg/day; or about 500 mg/day; or about 525 mg/day; or about 550 mg/day; or about 575 mg/day; or about 600 mg/day; or about 625 mg/day; or about 650 mg/day; or about 675 mg/day; or about 700 mg/day; or about 725 mg/day; or about 750 mg/day; or about 775 mg/day; or about 800 mg/day; or about 825 mg/day; or about 850 mg/day; or about 875 mg/day; or about 900 mg/day; or about 925 mg/day; or about 950 mg/day; or about 975 mg/day; or about 1,000 mg/day and including any ranges between any two numbers provided herein. The exact dose employed is determined by the attending clinician based on factors such as the age, weight, sex of the patient and the extent of progression of the oxidative ocular disease.

In one embodiment, the methods described herein employ compositions comprising deuterated docosahexaenoic acid or ester thereof with at least about 80 percent replacement of hydrogen of all of the bis-allylic sites with deuterium and with an average deuteration at the mono-allylic sites of from about 1 to about 35 percent based on all of the available mono-allylic sites. Such compositions are suitable for use in the dosing protocols described herein. The inclusion of deuterium in the bis-allylic sites stabilizes the deuterated docosahexaenoic acid against oxidative damage. This, in turn, stops the cascade of lipid peroxidation (LPO) thereby minimizing damage to the retinal cells. When concentrations of this deuterated docosahexaenoic acid reach a therapeutic level in the retina, the disease progression is significantly attenuated. In addition, the level of deuteration at the mono-allylic sites necessarily correlates to deuteration at the bis-allylic sites and evidence that deuteration during the synthesis is progressing to high levels at the bis-allylic sites. Moreover, the inclusion of deuterium in the deuterated docosahexaenoic acid has been found not to functionally interfere with or adversely affect the patient.

In one preferred embodiment, compositions used in the dosing protocol comprise a population of deuterated docosahexaenoic acids and/or esters thereof having on average at least 90% of the bis-allylic hydrogen atoms exchanged to deuterium atoms. Such compositions impart significant protection against LPO in vivo. In addition, the deuterated compositions contain a measurable amount of deuteration at the mono-allylic sites as well. In particular, the average level of hydrogen atoms exchanged for deuterium atoms at all mono-allylic sites ranges from about 1 to about 35% in the composition. Surprisingly, the inclusion of deuteration at the mono-allylic sites does not interfere with the protection accorded by deuteration at the bis-allylic sites.

In one embodiment, the onset of a therapeutic concentration is within 50 days from the start of treatment, preferably within 40 days, and more preferably, within 30 days.

In one embodiment, the periodic administration of docosahexaenoic acid or esters thereof comprises administration of a daily dose of docosahexaenoic acid or an ester thereof for at least 5 days per week during therapy. In another embodiment, the periodic administration of docosahexaenoic acid or esters thereof comprises administration of a daily dose of docosahexaenoic acid or an ester thereof once a day for 7 days per week during therapy.

In one embodiment, the rate of reduction in disease progression is based on the following formula:
a) determine the average rate of disease progression for a cohort of treated patients by measuring the extent of geographic atrophy in each of the patient's retina at the start of therapy and at 6 months or 12 months, or 18 months or 24 months post start of therapy, obtaining an average for that differential and assigning a first value to that average differential and assign “A” to that value;
b) determine the average rate of disease progression for a cohort of untreated patients by measuring the extent of geographic atrophy in each of the patient's retina at the start of therapy and at 6 months or 12 months, or 18 months or 24 months post start of therapy, obtaining an average for that differential and assigning a second value to that average differential and assign “B” to that value;
c) calculate the delta between A and B and assign “C” to that value;
d) assign a positive value to C if B is greater than A;
e) assign a negative value to C if A is greater than B; and
f) divide C by B and multiply by 100.

As an example, the following shows implementation of this calculation:
treated patients showed an average increase in the extent of geographic atrophy over 6 months of therapy of 0.15 ------ which is defined as A;
the placebo treated patients evidenced an average increase in the extent of geographic atrophy over 6 months of 0.24 ------ which is defined as B.
The differential between A and B is 0.09 which is assigned as “C”;
this value is assigned a positive number per d) above; and
divide C by B (0.09/0.24) and multiply by 100 to provide for 37.5 reduction in the rate of disease progression.

In one embodiment, this rate of reduction for a given patient is determined as above but by replacing A based on the cohort and using the result for the individual.

In one embodiment, the patients are placed on a diet that restricts intake of excessive amounts of PUFA compounds to maximize the uptake of the deuterated docosahexaenoic acid by the body. Generally, dietary components that contribute to excessive amounts of PUFA consumed are restricted. Such dietary components include, for example, fish oil pills, and salmon, and patients on conventional feeding tubes that result in excessive PUFA intake. In a preferred embodiment, the methods described herein include both the dosing regimen described above as well as placing the patients on a restrictive diet that avoids excessive ingestion of PUFA components.

### DETAILED DESCRIPTION

Disclosed are methods for treating oxidative ocular diseases that slow the rate of disease progression in a patient. In one embodiment, the methods of this invention include a dosing regimen that efficiently and rapidly provides a therapeutic level of deuterated docosahexaenoic acid in the eyes.

Prior to discussing this invention in more detail, the following terms will first be defined. Terms that are not defined are given their definition in context or are given their medically acceptable definition.

The terminology used herein is for the purpose of describing particular embodiments only and is not intended to be limiting of the invention. As used herein, the singular forms “a”, “an” and “the” are intended to include the plural forms as well, unless the context clearly indicates otherwise.

As used herein, the term “optional” or “optionally” means that the subsequently described event or circumstance can or cannot occur, and that the description includes instances where the event or circumstance occurs and instances where it does not.

As used herein, the term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to a dose amount means that the dose may vary by +/−10%.

As used herein, the term “comprising” or “comprises” is intended to mean that the compositions and methods include the recited elements, but not excluding others.

As used herein, the term “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention.

As used herein, the term “consisting of” shall mean excluding more than trace elements of other ingredients and substantial method steps. Embodiments defined by each of these transition terms are within the scope of this invention.

As used herein, the term docosahexaenoic acid refers to the compound having the known structure as follows:

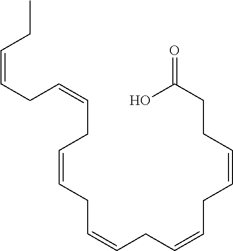 [SMILES: CC/C=C\C/C=C\C/C=C\C/C=C\C/C=C\C/C=C\CCC(=O)O]

Esters of docosahexaenoic acid are formed by replacing the —OH group with —OR. Such esters are as defined herein below.

As used herein and unless the context dictates otherwise, the term “deuterated docosahexaenoic acid or an ester thereof” refers to docosahexaenoic acid or ester compounds having on average at least 80 percent of the hydrogen atoms at the bis-allylic sites exchanged to deuterium atoms with on average no more than about 35 percent of the hydrogen atoms at the mono-allylic sites exchanged to deuterium atoms. In preferred embodiments, the average of hydrogen atoms at the bis-allylic sites exchanged to deuterium and the average of hydrogen atoms at the mono-allylic sites exchanged to deuterium are provided below

In one embodiment, deuterated DHA as described herein can be represented by formula I:

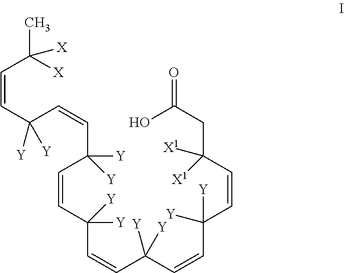

where each Y is independently hydrogen or deuterium provided that at least about 80% of all of said Y groups are deuterium; and
each X and X¹ is independently hydrogen or deuterium provided that the aggregate of all X and X¹ groups contain at least about 1% to about 35% of deuterium including all subranges found there between.

In one embodiment, the aggregate of both X groups contains from about 5% to about 30% of deuterium including all subranges between these two numbers whereas the aggregate of both X¹ groups contains from about 1% to about 10% of deuterium including all subranges between these two numbers.

Exemplary deuterated DHA compositions described herein are provided in Table 1 below and referencing Formula I above:

TABLE 1

| | Average percent | Average percent | Average percent |
|---|---|---|---|
| Exam- | deuterium at the | deuterium at both X | deuterium at both X¹ |
| ple | bis-allylic sites | mono-allylic sites | mono-allylic sites |
| 1 | at least about | from about 15 to | from about 1 to |
| | 80% | about 35% | about 15% |
| 2 | at least about | from about 15 to | from about 2 to |
| | 85% | about 30% | about 15% |
| 3 | at least about | from about 15 to | from about 3 to |
| | 85% | about 25% | about 15% |
| 4 | at least about | from about 10 to | from about 2 to |
| | 90% | about 25% | about 12% |
| 5 | at least about | from about 10 to | from about 2 to |
| | 90% | about 20% | about 10% |
| 6 | at least about | from about 5 to | from about 2 to |
| | 95% | about 20% | about 10% |
| 7 | at least about | from about 5 to | from about 2 to |
| | 95% | about 15% | about 10% |

As used herein and unless the context dictates otherwise, the term “an ester thereof” refers to a C₁-C₆ alkyl ester, glycerol ester (including monoglycerides, diglycerides and triglycerides), sucrose esters, phosphate esters, and the like. The particular ester employed is not critical provided that the ester is pharmaceutically acceptable (non-toxic and biocompatible).

As used herein, the term “phospholipid” refers to any and all phospholipids that are components of the cell membrane. Included within this term are phosphatidylcholine, lysophosphatydylcholine, phosphatidylethanolamine, phosphatidylserine, and sphingomyelin.

As used herein, the term “patient” refers to a human patient or a cohort of human patients suffering from an oxidative retinal disease treatable by administration of a compositions comprising deuterated docosahexaenoic acid or an ester thereof.

As used herein, the term “pharmaceutically acceptable salts” of compounds disclosed herein are within the scope of the methods described herein and include acid or base addition salts which retain the desired pharmacological activity and is not biologically undesirable (e.g., the salt is not unduly toxic, allergenic, or irritating, and is bioavailable). When the compound has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, trimethylamine, pyridine, picoline, ethanolamine, diethanolamine, triethanolamine) or basic amino acids (e.g., arginine, lysine, and ornithine). Such salts can be prepared in situ during isolation and purification of the compounds or by separately reacting the purified compound in its free base or free acid form with a suitable acid or base, respectively, and isolating the salt thus formed.

### Compound Preparation

Deuterated docosahexaenoic acid is prepared by the synthetic methods set forth in U.S. Pat. No. 10,730,821 which is incorporated herein by reference in its entirety. Specifically, Table 1 of that patent illustrates a single run for the synthetic protocol described therein that provided for 96% on average exchange of deuterium at the bis-allylic sites with about 26% on average deuteration at the mono-allylic sites.

Esters of these deuterated fatty acids are prepared by conventional techniques well known in the art.

### Methodology

The methods described herein entail the sustained dosing levels described herein to both achieve a therapeutic concentration and to maintain such a concentration in the eye and, specifically, in the rods of the retina. The dosing employed herein accounts for the variability of individual patients' metabolism with regard to the daily maximum PUFA uptake, the percent that the deuterated docosahexaenoic acid or ester thereof constitutes part of the PUFA uptake, specific conditions that compromise the PUFA uptake, and other factors well known in the art. In addition, the gradual increase in the in vivo concentration of docosahexaenoic acid and its relatively long half-life allows for patient medication “holidays” provided that the drug is administered at least 70% of the days per month, such as 5 days per week, 6 days per week, as well as 7 days per week for 3 weeks out of 4. In one embodiment, the drug is administered at least 85% of the days per month (e.g., at least six days per week). Therefore, a patient who intentionally or inadvertently misses a daily dose of the drug is still compliant with the overall dosing protocol which is quite dissimilar to conventional drugs.

The dosing regimen employs a daily or unit dose of from about 100 mg/day to about 1,000 mg/day without regard to the patient's BMI, severity of the disease condition, or the otherwise overall health of the patient. In one embodiment, the daily dose is from about 100 mg/day to about 350 mg/day. In another embodiment, the daily dose is from about 350 mg/day to about 650 mg/day. In yet another embodiment, the daily dose is from about 650 mg/day to about 1,000 mg/day. In specific examples, the deuterated docosahexaenoic acid or ester thereof is administered at about 100 mg/day; or about 125 mg/day; or about 150 mg/day; or about 175 mg/day; or about 200 mg/day; or about 225 mg/day; or about 250 mg/day; or about 275 mg/day; or about 300 mg/day; or about 325 mg/day; or about 350 mg/day; or about 375 mg/day, or about 400 mg/day; or about 425 mg/day; or about 450 mg/day; or about 475 mg/day; or about 500 mg/day; or about 525 mg/day; or about 550 mg/day; or about 575 mg/day; or about 600 mg/day; or about 625 mg/day; or about 650 mg/day; or about 675 mg/day; or about 700 mg/day; or about 725 mg/day; or about 750 mg/day; or about 775 mg/day; or about 800 mg/day; or about 825 mg/day; or about 850 mg/day; or about 875 mg/day; or about 900 mg/day; or about 925 mg/day; or about 950 mg/day; or about 975 mg/day; or about 1,000 mg/day. The dose administered may be any value or subrange within the recited ranges.

The diagnosis and progression of the oxidative ocular disease is evaluated by any one of a number of conventional diagnostic tools well known in the art. See, e.g., verywellhealth.com/how-macular-degeneration-is-diagnosed-4160590. In one embodiment, the rate of reduction in a patient's disease progression is evaluated by comparing the ocular tests results subsequent to start of therapy to those obtained at the time of the original diagnosis/start of therapy or to the test results from any prior evaluation. The data suggest that the rate of disease progression in an individual patient will be reduced by at least about 20%, or at least about 30%, or at least about 40%, or at least about 50% or more when the dosing methods described herein are employed. The amount of reduction may be any value or subrange within the recited ranges, including endpoints. In general, the comparison is between the known rate of disease progression and that experienced by the patient and is made at any time from 1 to 24 months, such as about 6, or 12, or 18, or 24 months after initiation of therapy and then periodically thereafter (e.g., every 6 months). In one embodiment, the known rate of disease progression can be based on the rate of geographic atrophy progression in a cohort of patients treated with placebo over the same period of time.

In another embodiment, the efficacy of the treatment protocol can be evaluated by comparing the extent of geographic atrophy progression in a treated population or individual against a placebo population. In such a comparison, efficacy is established by a statistically significant reduction in geographic atrophy progression in the treated population as compared to the placebo population. Preferably, the degree of reduction is at least by at least about 20%, or at least about 30%, or at least about 40%, or at least about 50% or more when the dosing methods described herein are employed.

The methods described herein are also based, in part, on the discovery that when the lipid membrane of the retinal cells is stabilized against LPO, there is a substantial reduction in the progression of the oxidative retinal disease. Without being limited by theory, it is believed this is because replacement of hydrogen atoms with deuterium atoms at the deuterated docosahexaenoic acid renders these carbon-deuterium bonds significantly more stable to ROS than the carbon-hydrogen atoms. As above, this stability manifests itself in reducing the cascade of lipid auto-oxidation and, hence, limiting the rate of disease progression.

### Combinations

The therapy provided herein can be combined with conventional treatment of used with oxidative retinal provided that such therapy is operating on an orthogonal mechanism of action relative to inhibition of lipid auto-oxidation. Suitable drugs for use in combination include, but not limited to, antioxidants such as edaravone, idebenone, mitoquinone, mitoquinol, vitamin C, or vitamin E provided that none of these antioxidants that are directed to inhibiting lipid auto-oxidation, riluzole which preferentially blocks TTX-sensitive sodium channels, conventional pain relief mediations, and the like.

### Pharmaceutical Compositions

The specific dosing of deuterated docosahexaenoic acid or an ester thereof is accomplished by any number of the accepted modes of administration. As noted above, the actual amount of the drug used in a daily or periodic dose per the methods of this invention, i.e., the active ingredient, is described in detail above. The drug can be administered at least once a day, preferably once or twice or three or more times a day.

This invention is not limited to any particular composition or pharmaceutical carrier, as such may vary. In general, compounds of this invention will be administered as pharmaceutical compositions by any of a number of known routes of administration. However, orally delivery is preferred typically using tablets, pills, capsules, and the like. The particular form used for oral delivery is not critical but due to the large amount of drug to be administered, a daily or periodic unit dose is preferably divided into subunits having a number of tablets, pills, capsules, and the like. In one particularly preferred embodiment, the docosahexaenoic acid or an ester thereof is administered in a gel capsule as a neat oil.

Pharmaceutical dosage forms of a compound of this invention may be manufactured by any of the methods well-known in the art, such as, by conventional mixing, tableting, encapsulating, and the like. The compositions of this invention can include one or more physiologically acceptable inactive ingredients that facilitate processing of active molecules into preparations for pharmaceutical use.

The compositions can comprise the drug in combination with at least one pharmaceutically acceptable excipient. Acceptable excipients are non-toxic, aid administration, and do not adversely affect the therapeutic benefit of the claimed compounds. Such excipient may be any solid, liquid, or semi-solid that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Other suitable pharmaceutical excipients and their formulations are described in Remington's Pharmaceutical Sciences, edited by E. W. Martin (Mack Publishing Company, 18th ed., 1990).

The compositions of this invention may, if desired, be presented in a pack or dispenser device each containing a daily or periodic unit dosage containing the drug in the required number of subunits. Such a pack or device may, for example, comprise metal or plastic foil, such as a blister pack, a vial, or any other type of containment. The pack or dispenser device may be accompanied by instructions for administration including, for example, instructions to take all of the subunits constituting the daily or periodic dose contained therein.

The amount of the drug in a formulation can vary depending on the number of subunits required for the daily or periodic dose of the drug. Typically, the formulation will contain, on a weight percent (wt %) basis, from about 10 to 100 weight percent of the drug based on the total formulation outside of the weight of the capsule carrier with the balance being one or more suitable pharmaceutical excipients. Preferably, the compound is present at a level of about 50 to 99 weight percent.

In preferred embodiment, the drug is encapsulated inside a capsule without the need for any pharmaceutical excipients such as stabilizers, antioxidants, colorants, etc.

### Examples

This invention is further understood by reference to the following examples, which are intended to be purely exemplary of this invention. This invention is not limited in scope by the exemplified embodiments, which are intended as illustrations of single aspects of this invention only. Any methods that are functionally equivalent are within the scope of this invention. Various modifications of this invention in addition to those described herein will become apparent to those skilled in the art from the foregoing description and accompanying figures. Such modifications fall within the scope of the appended claims. In these examples, the following terms are used herein and have the following meanings.

### Example 1—Preparation of Deuterated Docosahexaenoic Acid Ethyl Ester

Following the procedure of U.S. Pat. No. 10,730,821, a composition comprising docosahexaenoic acid ethyl ester was prepared which was deuterated at the bis-allylic positions at a level of greater than 80% on average and at the mono-allylic positions at less than 35% on average.

### Example 2—Reduction in the Rate of Disease Progression

This example illustrates the reduction in the rate of macular degeneration progression in a cohort of patients treated with deuterated docosahexaenoic acid ethyl ester similar to that of Example 1, as compared to a cohort of placebo patients. Specifically, the treated cohort is administered 250 mg/day of deuterated docosahexaenoic acid ethyl ester or 250 mg/day of safflower oil. The patients are maintained on this dosing regimen throughout the clinical study. Periodic measurements of further geographic atrophy development are obtained.

Dosing is continued for 6 or 12, or 18 or 24 months. At that time, the average extent of geographic atrophy progression is measured for each cohort. The efficacy of the treatment protocol is evaluated by comparing the extent of geographic atrophy progression in a treated population against a placebo population. Specifically, the methods describe herein provide a statistically significant reduction in the rate of disease progression.

### Example 3—Determination in the Reduction in Disease Progression Using Cohorts of Treated and Untreated Patients

In this example, the reduction in disease progression is determined as follows:
a) determine the average rate of disease progression for a cohort of patients treated with the deuterated docosahexaenoic acid ethyl ester by measuring the extent of geographic atrophy in each of the patient's retina at the start of therapy and at 6, 12, 18 or 24 months post start of therapy, determining the difference between the extent of atrophy at the start of therapy and at the later time point, and then obtaining an average for that differential and assigning a first value designated as “A” to that average differential;
b) determine the average rate of disease progression for a cohort of patients treated with placebo (safflower oil) by measuring the extent of geographic atrophy in each of the patient's retina at the start of therapy and at 6, 12, 18 or 24 months post start of therapy, determining the difference between the extent of atrophy at the start of therapy and at the later time point, and then obtaining an average for that differential and assigning a second value designated as “B” to that average differential;
c) calculating the difference between B and A and assign “C” to that value (e.g, B −A=C);
d) assign a positive value to “C” if B is greater than A;
e) assign a negative value to “C” if B is less than A; and
f) divide C by B and multiply by 100 [(C/B)×100].

As per this example, treated patients will have a positive percent reduction in geographic atrophy that is statistically significant and preferably at least a positive 20 percent reduction. That is to say that if A has an arbitrary value of 40 and B has an arbitrary value of 60, then B−A=C gives a value for C of 20. Then dividing C/B gives 20/60 and multiplying that value by 100=33%.

### Example 4—Determination in the Reduction in Disease Progression Using a Treated Patient and a Cohort of Untreated Patients

Alternatively, the rate of disease progression for an individual patient can be assessed by the following:
a) determine the rate of disease progression for said individual patient by measuring the extent of geographic atrophy in the patient's retina at the start of therapy and at 6, 12, 18 or 24 months post start of therapy and assigning a third value “D” to that differential;
b) determine the average rate of disease progression for a cohort of patients treated with placebo (safflower oil) by measuring the extent of geographic atrophy in each of the patient's retina at the start of therapy and at 6, 12, 18 or 24 months post start of therapy, determining the difference between the extent of atrophy at the start of therapy and at the later time point, and then obtaining an average for that differential and assigning a second value designated as “E” to that average differential;
c) calculating the difference between D and E and assign “F” to that value (e.g, E−D=F);
d) assign a positive value to “F” if E is greater than D;
e) assign a negative value to “F” if E is less than D; and
f) divide F by E and multiply by 100 [(F/E)×100].
   As per this example, a treated patient will have a positive percent reduction in geographic atrophy that is statistically significant and preferably at least a positive 20 percent reduction. That is to say that if D has an arbitrary value of 50 and E has an arbitrary value of 100, then E−D=F gives a value for F of 50. Then dividing F/E gives 50/100 and multiplying that value by 100=50%.

1. A method for reducing a rate of disease progression for an oxidative retinal disease in a retina of a patient, said method comprising periodic administration to said patient of from about 100 mg/day to about 1,250 mg/day amount of a composition comprising deuterated docosahexaenoic acid or ester thereof, wherein said administration results in a therapeutic concentration of deuterated docosahexaenoic acid in the retina coupled with the reduction in a rate of progression of said oxidative retinal disease.

2. The method of claim 1, wherein said periodic administration of the composition comprises administration for at least 5 days per week.

3. The method of claim 1, wherein said deuterated docosahexaenoic acid or ester thereof in the composition comprises an average deuteration at bis-allylic sites of at least about 80 percent based on all of the available bis-allylic sites, and wherein an average deuteration at mono-allylic sites is from about 1 percent to about 35 percent based on all of the available mono-allylic sites.

4. The method of claim 3, wherein said average deuteration at bis-allylic sites is at least about 90 percent and the average deuteration at the mono-allylic sites is from about 1 percent to about 25 percent.

5. The method of claim 1, wherein the onset of a therapeutic concentration of docosahexaenoic acid in the retina of the patient is within 50 days from the start of treatment.

6. The method of claim 5, wherein the onset of a therapeutic concentration of docosahexaenoic acid in the retina of the patient is within 40 days from the start of treatment.

7. The method of claim 5, wherein the onset of a therapeutic concentration of docosahexaenoic acid in the retina of the patient is within 30 days from the start of treatment

8. The method of claim 1, wherein the reduction in the rate of progression is compared to an average rate of reduction in disease progression in a cohort of treated patients as determined by the average reduction in the rate of progression of said oxidative retinal disease in the cohort of treated patient as compared to an average rate of progression in a cohort of placebo-treated patients employing the formula of Example 2.

9. The method of claim 1, wherein the average rate of reduction in disease progression in a treated patient is determined by his or her reduction in the rate of progression of said oxidative retinal disease as compared to an average rate of progression in a cohort of placebo-treated patients employing the formula of Example 3.

10. The method of claim 8, wherein day 0 is prior to treatment with deuterated docosahexaenoic acid or ester thereof.

11. The method of claim 9, wherein day 0 is prior to treatment with placebo.

12. The method of claim 8, wherein the time period is 6 months or 12 months.

13. The method of claim 1, wherein the reduction in the rate of progression of said oxidative retinal disease in the patient is determined based on comparison with a rate of progression in the patient's retina prior to treatment.

14. The method of claim 1, wherein said ester is a C₁-C₆ alkyl ester, a monoglyceride ester, a diglyceride ester, a triglyceride ester, a sucrose ester, or a phosphate ester.

15. The method of claim 14, wherein said ester is an ethyl ester.

16. The method of claim 1, further comprising placing the patient on a diet that restricts intake of non-deuterated polyunsaturated fatty acids.
